(19) United States
(12) Patent Application Publication
(10) Pub. No.: US 2025/0037484 A1
(43) Pub. Date: Jan. 30, 2025

(54) SYSTEMS AND METHODS FOR DIGITIZING A SLIDE

(71) Applicant: Pramana, Inc., Cambridge, MA (US)

(72) Inventors: Prasanth Perugupalli, Cary, NC (US); Jaya Jain, Bhopal (IN); Prateek Jain, Karnataka (IN); Durgaprasad Dodle, Telangana (IN); Deepak Anand, Karnataka (IN); Raghubansh Bahadur Gupta, Bangalore (IN); Asa Rubin, Silver Springs, MD (US)

(73) Assignee: Pramana, Inc., Cambridge, MA (US)

(21) Appl. No.: 18/640,483

(22) Filed: Apr. 19, 2024

Related U.S. Application Data
(63) Continuation-in-part of application No. 18/227,155, filed on Jul. 27, 2023, now Pat. No. 11,997,240.

(51) Int. Cl. G06V 20/69; G06V 10/82; G06V 30/148; G16H 10/60

(52) U.S. Cl. CPC G06V 20/693; G06V 10/82; G06V 30/153; G16H 10/60

(57) ABSTRACT

Described herein are systems and methods for digitizing a slide. A system may include a slide; an optical system; and a computing device configured to identify metadata associated with the slide; determine a first scanning parameter as a function of the metadata; and using the optical system, capture a z-stack as a function of the first scanning parameter.

### CROSS-REFERENCE TO RELATED APPLICATIONS

This application is a continuation-in-part of Non-provisional application Ser. No. 18/227,155 filed on Jul. 27, 2023, and entitled “METHOD AND AN APPARATUS FOR INLINE IMAGE SCAN ENRICHMENT,” the entirety of which is incorporated herein by reference.

### FIELD OF THE INVENTION

The present invention generally relates to the field of slide digitization. In particular, the present invention is directed to systems and methods for digitizing a slide.

### BACKGROUND

Digitization of images at scale in healthcare, an activity that is still in its early stages, is central to improving patient healthcare, given the wealth of information contained in them complementing other modalities. A key factor to scale this process is the current inefficiency in the digitization process. Digitization is currently done as a two-stage process with scanning as the first step and image processing as the second step. This two-stage approach suffers from a critical drawback-issues during the scanning process are not detected. This forces a rescan of those images once they are detected in the second stage of image processing.

### SUMMARY OF THE DISCLOSURE

In an aspect, a system for digitizing a slide may include a slide; an optical system; and a computing device configured to identify metadata associated with the slide; determine a first scanning parameter as a function of the metadata; and using the optical system, capture a z-stack as a function of the first scanning parameter.

In another aspect, a method of digitizing a slide may include, using at least a processor, identifying metadata associated with the slide; using the at least a processor, determining a first scanning parameter as a function of the metadata; and using an optical system and the at least a processor, capturing a z-stack as a function of the first scanning parameter.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a diagram depicting an exemplary embodiment of a system for digitizing a slide;

FIG. 2 is a diagram depicting an exemplary embodiment of a system for digitizing a slide;

FIG. 3 is a box diagram of an exemplary machine learning model;

FIG. 4 is a diagram of an exemplary neural network;

FIG. 5 is a diagram of an exemplary neural network node;

FIG. 6 is a flow diagram depicting an exemplary embodiment of a method of digitizing a slide;

FIG. 7 is a flow diagram depicting an exemplary embodiment of a method of digitizing a slide; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for digitizing a slide. A method may include inline enrichment of images during scanning, which may be used to eliminate errors of a two-stage process of scanning and subsequent image processing. Specifically, a method may perform hardware and/or software adjustments during scanning to optimally scan an image, maximizing the capture of information useful for downstream diagnosis and decision-making. A method may include utilization of information that is either present on the slide in text form or annotations and/or metadata available from subject electronic health records during a scan process to maximize detection of information critical to downstream diagnosis and/or decision making. A method may create a stack of scans that enables downstream creation of a continuous viewing experience at different focal points by interpolating in latent space of a diffusion model between scanned stack positions. In some embodiments, this approach may reduce the likelihood of scanned images failing to capture key attributes which may be required by a downstream process.

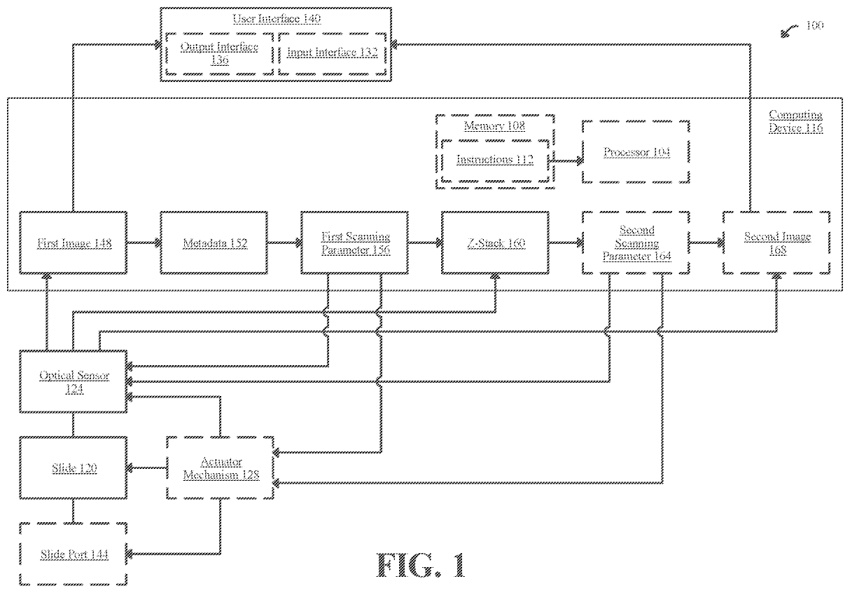

Referring now to FIG. 1, an exemplary embodiment of a system 100 for digitizing a slide is illustrated. System 100 may include a computing device. System 100 may include a processor. Processor may include, without limitation, any processor described in this disclosure. Processor may be included in computing device. Computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device.

Still referring to FIG. 1, in some embodiments, system 100 may include at least a processor 104 and a memory 108 communicatively connected to the at least a processor 104, the memory 108 containing instructions 112 configuring the at least a processor 104 to perform one or more processes described herein. Computing device 116 may include processor 104 and/or memory 108. Computing device 116 may be configured to perform one or more processes described herein.

Still referring to FIG. 1, computing device 116 may include but is not limited to, for example, a computing device or cluster of computing devices in a first location and a second computing device or cluster of computing devices in a second location. Computing device 116 may include one or more computing devices dedicated to data storage, security, distribution of traffic for load balancing, and the like. Computing device 116 may distribute one or more computing tasks as described below across a plurality of computing devices of computing device, which may operate in parallel, in series, redundantly, or in any other manner used for distribution of tasks or memory between computing devices. Computing device 116 may be implemented, as a non-limiting example, using a “shared nothing” architecture.

With continued reference to FIG. 1, computing device 116 may be designed and/or configured to perform any method, method step, or sequence of method steps in any embodiment described in this disclosure, in any order and with any degree of repetition. For instance, computing device 116 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 116 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, as used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, cither directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Still referring to FIG. 1, in some embodiments, system 100 may be used to generate an image of slide 120 and/or a sample on slide. As used herein, a “slide” is a container or surface holding a sample of interest. In some embodiments, slide 120 may include a glass slide. In some embodiments, slide may include a formalin fixed paraffin embedded slide. In some embodiments, a sample on slide may be stained. In some embodiments, slide 120 may be substantially transparent. In some embodiments, slide 120 may include a thin, flat, and substantially transparent glass slide. In some embodiments, a transparent cover may be applied to slide such that a sample is between slide and this cover. A sample may include, in non-limiting examples, a blood smear, pap smear, body fluids, and non-biologic samples. In some embodiments, sample on slide 120 may include a biological sample. In some embodiments, a sample on slide 120 may include tissue. In some embodiments, sample on slide 120 may be frozen.

Still referring to FIG. 1, in some embodiments, slide 120 and/or a sample on slide may be illuminated. In some embodiments, system 100 may include a light source. As used herein, a “light source” is any device configured to emit electromagnetic radiation. In some embodiments, light source may emit a light having substantially one wavelength. In some embodiments, light source may emit a light having a wavelength range. Light source may emit, without limitation, ultraviolet light, visible light, and/or infrared light. In non-limiting examples, light source may include a light-emitting diode (LED), an organic LED (OLED) and/or any other light emitter. Such a light source may be configured to illuminate slide 120 and/or sample on slide 120. In a non-limiting example, light source may illuminate slide 120 and/or sample on slide 120 from below.

Still referring to FIG. 1, in some embodiments, system 100 may include at least an optical system. As used in this disclosure, an “optical system” is an arrangement of one or more components which together detect electromagnetic radiation. In non-limiting examples, electromagnetic radiation may include light, such as visible light, infrared light, UV light, and the like. An optical system may include one or more optical elements, including without limitation lenses, mirrors, windows, filters, and the like. An optical system may form an optical image that corresponds to an optical object. For instance, an optical system may form an optical image at or upon an optical sensor, which can capture, e.g., digitize, the optical image. In some cases, optical system may have at least a magnification. For instance, optical system may include an objective (e.g., microscope objective) and one or more reimaging optical elements that together produce an optical magnification. In some cases, optical magnification may be referred to herein as zoom. In some embodiments, an optical system may include optical sensor 124. In some embodiments, an optical system may include a plurality of optical sensors. As used herein, an “optical sensor” is a device that measures light and converts the measured light into one or more signals. One or more signals may include, without limitation, one or more electrical signals. In some embodiments, optical sensor may include a photodetector. In some embodiments, a photodetector may include a photodiode, a photoresistor, a photosensor, a photovoltaic chip, and the like. In some embodiments, optical sensor may include a plurality of photodetectors. Optical sensor may include, without limitation, a camera. Optical sensor may be in electronic communication with at least a processor of system 100. As used herein, “electronic communication” as used in this disclosure is a shared data connection between two or more devices. In some embodiments, system 100 may include two or more optical sensors.

Still referring to FIG. 1, in some embodiments, optical system may include a camera. In some cases, a camera may include one or more optics. Exemplary non-limiting optics include spherical lenses, aspherical lenses, reflectors, polarizers, filters, windows, aperture stops, and the like. In some embodiments, one or more optics associated with a camera may be adjusted in order to, in non-limiting examples, change the zoom, depth of field, and/or focus distance of the camera. In some embodiments, one or more of such settings may be configured to detect a feature of a sample on slide 120. In some embodiments, one or more of such settings may be configured based on a scanning parameter, as described herein. In some embodiments, camera may capture images at a low depth of field. In a non-limiting example, camera may capture images such that a first depth of sample is in focus and a second depth of sample is out of focus. In some embodiments, an autofocus mechanism may be used to determine focus distance. In some embodiments, focus distance may be set by scanning parameter. In some embodiments, camera may be configured to capture a plurality of images at different focus distances. In a non-limiting example, camera may capture a plurality of images at different focus distances, such that images are captured where each sample focus depth of the sample is in focus in at least one image. In some embodiments, at least a camera may include an image sensor. Exemplary non-limiting image sensors include digital image sensors, such as without limitation charge-coupled device (CCD) sensors and complimentary metal-oxide-semiconductor (CMOS) sensors. In some embodiments, a camera may be sensitive within a non-visible range of electromagnetic radiation, such as without limitation infrared.

Still referring to FIG. 1, as used herein, “image data” is information representing at least a physical scene, space, and/or object. Image data may include, for example, information representing a sample, slide 120, or region of a sample or slide. In some cases, image data may be generated by a camera. An image may be optical, such as without limitation where at least an optic is used to generate an image of an object. An image may be digital, such as without limitation when represented as a bitmap. Alternatively, an image may include any media capable of representing a physical scene, space, and/or object. “Image data” may be used interchangeably through this disclosure with “image,” where image is used as a noun. Where “image” is used as a verb in this disclosure, it refers to generation and/or formation of an image.

Still referring to FIG. 1, in some embodiments, system 100 may include a slide port 144. In some embodiments, slide port 144 may be configured to hold slide 120. In some embodiments, slide port 144 may include one or more alignment features. As used herein, an “alignment feature” is a physical feature that helps to secure a slide in place, align a slide with another component, or both. In some embodiments, alignment feature may include a component which keeps slide secure, such as a clamp, latch, clip, recessed area, or another fastener. In some embodiments, slide port may allow for easy removal or insertion of slide. In some embodiments, slide port 144 may include a transparent surface through which light may travel. In some embodiments, slide may rest on and/or may be illuminated by light traveling through such a transparent surface. In some embodiments, slide port may be mechanically connected to an actuator mechanism as described below.

Still referring to FIG. 1, in some embodiments, system 100 may include an actuator mechanism 128. As used herein, an “actuator mechanism” is a mechanical component configured to change the relative position of a slide and an optical system. In some embodiments, actuator mechanism 128 may be mechanically connected to slide 120. In some embodiments, actuator mechanism may be mechanically connected to slide port 144. For example, actuator mechanism 128 may move slide port 144 in order to move slide 120. In some embodiments, actuator mechanism 128 may be mechanically connected to at least an optical system. In some embodiments, actuator mechanism may be mechanically connected to a mobile element. A mobile element may include a movable or portable object, component, and/or device within system 100 such as, without limitation, a slide, a slide port, or an optical system. In some embodiments, a mobile element may move such that optical system is positioned correctly with respect to slide such that optical system may capture an image of slide according to a scanning parameter. In some embodiments, actuator mechanism may be mechanically connected to an item selected from the list consisting of slide port, slide, and at least an optical system. In some embodiments, actuator mechanism may be configured to change the relative position of slide and optical system by moving slide port, slide, and/or optical system.

Still referring to FIG. 1, actuator mechanism 128 may include a component of a machine that is responsible for moving and/or controlling a mechanism or system. Actuator mechanism may, in some embodiments, require a control signal and/or a source of energy or power. In some cases, a control signal may be relatively low energy. Exemplary control signal forms include electric potential or current, pneumatic pressure or flow, or hydraulic fluid pressure or flow, mechanical force/torque or velocity, or even human power. In some cases, an actuator may have an energy or power source other than control signal. This may include a main energy source, which may include for example electric power, hydraulic power, pneumatic power, mechanical power, and the like. In some embodiments, upon receiving a control signal, actuator mechanism responds by converting source power into mechanical motion. In some cases, actuator mechanism may be understood as a form of automation or automatic control.

Still referring to FIG. 1, in some embodiments, actuator mechanism 128 may include a hydraulic actuator. A hydraulic actuator may consist of a cylinder or fluid motor that uses hydraulic power to facilitate mechanical operation. Output of hydraulic actuator mechanism may include mechanical motion, such as without limitation linear, rotatory, or oscillatory motion. In some embodiments, hydraulic actuator may employ a liquid hydraulic fluid. As liquids, in some cases, are incompressible, a hydraulic actuator can exert large forces. Additionally, as force is equal to pressure multiplied by area, hydraulic actuators may act as force transformers with changes in area (e.g., cross sectional area of cylinder and/or piston). An exemplary hydraulic cylinder may consist of a hollow cylindrical tube within which a piston can slide. In some cases, a hydraulic cylinder may be considered single acting. Single acting may be used when fluid pressure is applied substantially to just one side of a piston. Consequently, a single acting piston can move in only one direction. In some cases, a spring may be used to give a single acting piston a return stroke. In some cases, a hydraulic cylinder may be double acting. Double acting may be used when pressure is applied substantially on each side of a piston; any difference in resultant force between the two sides of the piston causes the piston to move.

Still referring to FIG. 1, in some embodiments, actuator mechanism 128 may include a pneumatic actuator mechanism. In some cases, a pneumatic actuator may enable considerable forces to be produced from relatively small changes in gas pressure. In some cases, a pneumatic actuator may respond more quickly than other types of actuators, for example hydraulic actuators. A pneumatic actuator may use compressible fluid (e.g., air). In some cases, a pneumatic actuator may operate on compressed air. Operation of hydraulic and/or pneumatic actuators may include control of one or more valves, circuits, fluid pumps, and/or fluid manifolds.

Still referring to FIG. 1, in some cases, actuator mechanism 128 may include an electric actuator. Electric actuator mechanism may include any of electromechanical actuators, linear motors, and the like. In some cases, actuator mechanism may include an electromechanical actuator. An electromechanical actuator may convert a rotational force of an electric rotary motor into a linear movement to generate a linear movement through a mechanism. Exemplary mechanisms, include rotational to translational motion transformers, such as without limitation a belt, a screw, a crank, a cam, a linkage, a scotch yoke, and the like. In some cases, control of an electromechanical actuator may include control of electric motor, for instance a control signal may control one or more electric motor parameters to control electromechanical actuator. Exemplary non-limitation electric motor parameters include rotational position, input torque, velocity, current, and potential. Electric actuator mechanism may include a linear motor. Linear motors may differ from electromechanical actuators, as power from linear motors is output directly as translational motion, rather than output as rotational motion and converted to translational motion. In some cases, a linear motor may cause lower friction losses than other devices. Linear motors may be further specified into at least 3 different categories, including flat linear motor, U-channel linear motors and tubular linear motors. Linear motors may be directly controlled by a control signal for controlling one or more linear motor parameters. Exemplary linear motor parameters include without limitation position, force, velocity, potential, and current.

Still referring to FIG. 1, in some embodiments, actuator mechanism 128 may include a mechanical actuator mechanism. In some cases, a mechanical actuator mechanism may function to execute movement by converting one kind of motion, such as rotary motion, into another kind, such as linear motion. An exemplary mechanical actuator includes a rack and pinion. In some cases, a mechanical power source, such as a power take off may serve as power source for a mechanical actuator. Mechanical actuators may employ any number of mechanisms, including for example without limitation gears, rails, pulleys, cables, linkages, and the like.

Still referring to FIG. 1, in some embodiments, actuator mechanism 128 may be in electronic communication with actuator controls. As used herein, “actuator controls” is a system configured to operate actuator mechanism such that a slide and an optical system reach a desired relative position. In some embodiments, actuator controls may operate actuator mechanism based on input received from a user interface. In some embodiments, actuator controls may be configured to operate actuator mechanism such that optical system is in a position to capture an image of an entire sample. In some embodiments, actuator controls may be configured to operate actuator mechanism such that optical system is in a position to capture an image using settings of a particular scanning parameter. In some embodiments, actuator controls may be configured to operate actuator mechanism such that optical system is in a position to capture an image of a region of interest, a particular horizontal row, a particular point, a particular focus distance, and the like. Electronic communication between actuator mechanism and actuator controls may include transmission of signals. For example, actuator controls may generate physical movements of actuator mechanism in response to an input signal. In some embodiments, input signal may be received by actuator controls from processor or input interface.

Still referring to FIG. 1, in some embodiments, system 100 may include a user interface 140. User interface may include input interface 132 and output interface 136. In some embodiments, output interface 136 may include one or more elements through which system 100 may communicate information to a user. In a non-limiting example, output interface may include a display. A display may include a high resolution display. A display may output images, videos, and the like to a user. In another non-limiting example, output interface may include a speaker. A speaker may output audio to a user. In another non-limiting example, output interface may include a haptic device. A haptic device may output haptic feedback to a user.

Still referring to FIG. 1, in some embodiments, input interface 132 may include controls for operating system 100. Such controls may be operated by a user. Input interface may include, in non-limiting examples, a camera, microphone, keyboard, touch screen, mouse, joystick, foot pedal, button, dial, and the like. Input interface may accept, in non-limiting examples, mechanical input, audio input, visual input, text input, and the like. In some embodiments, input interface may approximate controls of a microscope.

Still referring to FIG. 1, in some embodiments, system 100 may capture first image 148 of slide 120. First image may be captured using an optical system as described above. In some embodiments, first image 148 may include a macro image. As used herein, a “macro image” is an image in which entirety of a sample on a slide is in frame. In an example, first image 148 may include an image of a region of a sample. A macro image may be a wider angle image than subsequently captured images. A macro image may include a lower resolution image than a subsequently captured image. A macro image may be used to determine subsequent scanning parameters, as described herein. In some embodiments, first image 148 may be captured as a function of a preset scanning parameter, a default scanning parameter, and/or a scanning parameter input by a user. Scanning parameters are described further below.

Still referring to FIG. 1, in some embodiments, system 100 identifies metadata 152 of first image 148. As used herein, “metadata” is a data structure that provides information about an image, information about a system depicted by an image, or both. In some embodiments, metadata 152 may describe a feature of an image data structure, such as the size in memory of the image data structure, the dimensions in pixels of an image, a format for encoding an image, and the like. In some embodiments, metadata 152 may describe the settings used to capture an image, such as the level of magnification used to capture an image, the aperture used to capture an image, the shutter speed used to capture an image, the ISO sensitivity used to capture an image, the resolution at which an image was captured, a level of backlighting, which of a plurality of optical sensors was used to capture an image, and the like. In some embodiments, metadata 152 may describe contextual information about circumstances present when an image was captured, such as the date an image was captured, the location an image was captured at, an identifier (such as a name or account name) describing a user who captured an image, an identifier describing an optical system used to capture an image, and the like. In some embodiments, metadata 152 may describe a feature of a system, such as slide 120, depicted by an image. As examples, metadata 152 may describe a subject from which a biological sample on slide 120 was obtained, a body part of subject from which a biological sample on slide 120 was obtained, a medical condition of such a subject, a description of a technique used to procure a biological sample on slide 120 from a subject, a mass, length, area, or volume of a biological sample on slide 120, a stain applied to a biological sample on slide 120, or a fixative or other chemical applied to a biological sample on slide 120. Metadata 152 may be associated with an image and/or set of images. In some embodiments, metadata 152 associated with an image of a slide may be determined before the image of the slide is captured. For example, metadata 152 may include a data structure describing a slide and/or a biological sample on a slide depicted in an image.

Still referring to FIG. 1, in some embodiments, metadata 152 includes data about data. Metadata 152 may help in understanding and managing various aspects of data, such as its origin, content, format, quality, and usage. Metadata 152 may play a crucial role in organizing, searching, and interpreting data effectively. Metadata 152 may include descriptive metadata, structural metadata, administrative metadata, technical metadata, provenance metadata, usage metadata, and the like. Metadata 152 may be organized and managed through metadata schemas, standards, or frameworks. These provide guidelines and specifications for capturing, storing, and exchanging metadata in a consistent and structured manner. Common metadata standards include Dublin Core, Metadata Object Description Schema (MODS), and the Federal Geographic Data Committee (FGDC) metadata standard. In some cases, metadata 152 may be associated with a label for a histology slide. Metadata 152 may provide additional descriptive information or attributes that are linked to the slide's label. This metadata 152 provides context and relevant details about the slide, aiding in its identification, categorization, and management within a pathology laboratory or medical facility. The specific metadata 152 associated with a label can vary based on the requirements and practices of the medical facility. Metadata 152 associated with the label may include patient information. Patient information may include data such as the patient's name, unique patient identifier (ID), age, gender, and any other relevant demographic information. Patient information helps in identifying and associating the slide with the correct individual's medical records. Metadata 152 may also include case specific details, wherein case specific details may include information about the specific case or clinical scenario related to the slide. Case specific details may include information about the case number, referring physician, clinical history, relevant symptoms, or any other pertinent details that aid in understanding the context of the slide. In some cases, metadata 152 may include information related to the specific specimen type of the slide. This may include the type of tissue or sample that the slide represents. Metadata 152 may contain notes, comments, or observations made by the pathologist or other medical professional. These annotations might highlight specific features, anomalies, or noteworthy aspects of the slide that are important for interpretation or follow-up analysis. For instance, a timestamp reflecting when and where the slide was prepared, analyzed, or labeled can be associated as metadata 152. This information helps in tracking and maintaining a chronological record of slide-related activities. It could be breast tissue, lung biopsy, skin lesion, or any other anatomical or pathological specimen. In some embodiments, metadata may contain information regarding staining or preparation technique, pathological diagnosis, and the like.

Still referring to FIG. 1, in some embodiments, metadata 152 may be recorded when an image is captured. For example, a user profile currently active and/or optical system settings may be recorded when an image is captured and may be stored as metadata of a captured image. For example, a time and/or location when an image is captured may be recorded and used as metadata. In some embodiments, metadata may be identified from a data structure associated with a slide, such as a subject profile or other information about a subject. For example, a subject identifier may be used to look up information relating to a subject and/or a biological sample, such as a procedure used to obtain the biological sample. For example, a biological sample feature may be identified from an electronic health record of a subject from which a biological sample was taken. As used herein, a “biological sample feature” is a data structure describing a biological sample, an aspect of a biological sample, or both. As examples, a biological sample feature may include a tissue type of a biological sample, and/or a mass of a biological sample. Information such as a biological sample feature may be retrieved from an electronic health record of a subject, which may be retrieved from a database containing electronic health records. As used herein, an “electronic health record” is a data structure describing a feature of a medical state of subject, a feature of a medical history of subject, or both. For example, an electronic health record may include a description of a biological sample, a procedure used to obtain biological sample, a date on which biological sample was taken, a medical condition subject had when biological sample was taken, and the like. An electronic health record may also include information such as age, gender, and/or ethnicity. In some embodiments, slide 120 may include a biological sample of a subject, and identifying metadata may include receiving an electronic health record of the subject. In some embodiments, an electronic health record may be obtained from a database storing electronic health records and/or a computing device associated with such a database. In some embodiments, an electronic health record may be determined from an image such as from an image of an annotation on a slide.

In some embodiments, metadata may be obtained as a function of an annotation on slide 120. For example, an annotation may include information such as an identifier describing which slide of a set of slides is present. Such an identifier may be associated with additional information such as an identifier which may be used to obtain an electronic health record. In some embodiments, metadata may include one or more coordinates on a slide at which an image is captured. In some embodiments, multiple images may be used to create an image assembled from the multiple images. In this context, the assembled image may include metadata of one or more of the images used to assemble it. In some embodiments, an initial image file may be used to create a subsequent image file. For example, the subsequent image file may be the result of an application of an image processing technique to the initial image file, and metadata of the initial image file may be used as metadata of the subsequent image file. Annotations on slide 120 may be read using optical character recognition (OCR).

Still referring to FIG. 1, in some embodiments, image data may be processed using optical character recognition. In some embodiments, optical character recognition or optical character reader (OCR) includes automatic conversion of images of written (e.g., typed, handwritten or printed text) into machine-encoded text. In some cases, recognition of at least a keyword from image data may include one or more processes, including without limitation optical character recognition (OCR), optical word recognition, intelligent character recognition, intelligent word recognition, and the like. In some cases, OCR may recognize written text, one glyph or character at a time. In some cases, optical word recognition may recognize written text, one word at a time, for example, for languages that use a space as a word divider. In some cases, intelligent character recognition (ICR) may recognize written text one glyph or character at a time, for instance by employing machine-learning processes. In some cases, intelligent word recognition (IWR) may recognize written text, one word at a time, for instance by employing machine-learning processes.

Still referring to FIG. 1, in some cases OCR may be an “offline” process, which analyses a static document or image frame. In some cases, handwriting movement analysis can be used as input to handwriting recognition. For example, instead of merely using shapes of glyphs and words, this technique may capture motions, such as the order in which segments are drawn, the direction, and the pattern of putting the pen down and lifting it. This additional information may make handwriting recognition more accurate. In some cases, this technology may be referred to as “online” character recognition, dynamic character recognition, real-time character recognition, and intelligent character recognition.

Still referring to FIG. 1, in some cases, OCR processes may employ pre-processing of image data. Pre-processing process may include without limitation de-skew, de-speckle, binarization, line removal, layout analysis or “zoning,” line and word detection, script recognition, character isolation or “segmentation,” and normalization. In some cases, a de-skew process may include applying a transform (e.g., homography or affine transform) to image data to align text. In some cases, a de-speckle process may include removing positive and negative spots and/or smoothing edges. In some cases, a binarization process may include converting an image from color or greyscale to black-and-white (i.e., a binary image). Binarization may be performed as a simple way of separating text (or any other desired image component) from a background of image data. In some cases, binarization may be required for example if an employed OCR algorithm only works on binary images. In some cases, a line removal process may include removal of non-glyph or non-character imagery (e.g., boxes and lines). In some cases, a layout analysis or “zoning” process may identify columns, paragraphs, captions, and the like as distinct blocks. In some cases, a line and word detection process may establish a baseline for word and character shapes and separate words, if necessary. In some cases, a script recognition process may, for example in multilingual documents, identify script allowing an appropriate OCR algorithm to be selected. In some cases, a character isolation or “segmentation” process may separate signal characters, for example character-based OCR algorithms. In some cases, a normalization process may normalize aspect ratio and/or scale of image data.

Still referring to FIG. 1, in some embodiments an OCR process may include an OCR algorithm. Exemplary OCR algorithms include matrix matching process and/or feature extraction processes. Matrix matching may involve comparing an image to a stored glyph on a pixel-by-pixel basis. In some case, matrix matching may also be known as “pattern matching,” “pattern recognition,” and/or “image correlation.” Matrix matching may rely on an input glyph being correctly isolated from the rest of image data. Matrix matching may also rely on a stored glyph being in a similar font and at a same scale as input glyph. Matrix matching may work best with typewritten text.

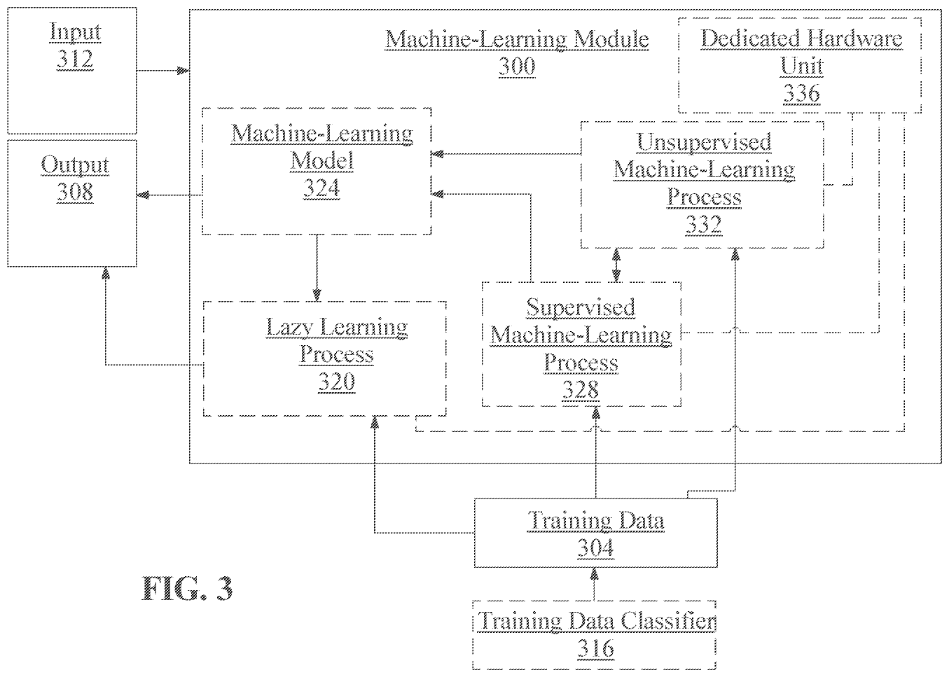

Still referring to FIG. 1, in some embodiments, an OCR process may include a feature extraction process. In some cases, feature extraction may decompose a glyph into at least a feature. Exemplary non-limiting features may include corners, edges, lines, closed loops, line direction, line intersections, and the like. In some cases, feature extraction may reduce dimensionality of representation and may make the recognition process computationally more efficient. In some cases, extracted feature may be compared with an abstract vector-like representation of a character, which might reduce to one or more glyph prototypes. General techniques of feature detection in computer vision are applicable to this type of OCR. In some embodiments, machine-learning processes like nearest neighbor classifiers (e.g., k-nearest neighbors algorithm) may be used to compare image features with stored glyph features and choose a nearest match. OCR may employ any machine-learning process described in this disclosure, for example machine-learning processes described with reference to FIGS. 3-5. Exemplary non-limiting OCR software includes Cuneiform and Tesseract. Cuneiform is a multi-language, open-source optical character recognition system originally developed by Cognitive Technologies of Moscow, Russia. Tesseract is free OCR software originally developed by Hewlett-Packard of Palo Alto, California, United States.

Still referring to FIG. 1, in some cases, OCR may employ a two-pass approach to character recognition. A first pass may try to recognize a character. Each character that is satisfactory is passed to an adaptive classifier as training data. The adaptive classifier then gets a chance to recognize characters more accurately as it further analyzes image data. Since the adaptive classifier may have learned something useful a little too late to recognize characters on the first pass, a second pass is run over the image data. Second pass may include adaptive recognition and use characters recognized with high confidence on the first pass to recognize better remaining characters on the second pass. In some cases, two-pass approach may be advantageous for unusual fonts or low-quality image data. Another exemplary OCR software tool include OCRopus. OCRopus development is led by German Research Centre for Artificial Intelligence in Kaiserslautern, Germany. In some cases, OCR software may employ neural networks.

Still referring to FIG. 1, in some cases, OCR may include post-processing. For example, OCR accuracy may be increased, in some cases, if output is constrained by a lexicon. A lexicon may include a list or set of words that are allowed to occur in a document. In some cases, a lexicon may include, for instance, all the words in the English language, or a more technical lexicon for a specific field. In some cases, an output stream may be a plain text stream or file of characters. In some cases, an OCR process may preserve an original layout of image data. In some cases, near-neighbor analysis can make use of co-occurrence frequencies to correct errors, by noting that certain words are often seen together. For example, “Washington, D.C.” is generally far more common in English than “Washington DOC.” In some cases, an OCR process may make us of a priori knowledge of grammar for a language being recognized. For example, grammar rules may be used to help determine if a word is likely to be a verb or a noun. Distance conceptualization may be employed for recognition and classification. For example, a Levenshtein distance algorithm may be used in OCR post-processing to further optimize results.

Still referring to FIG. 1, in some embodiments, metadata 152 may provide context, details, and parameters related to the scanning process and the scanned slide. In some cases, metadata 152 associated with an image may include one or more timestamps (e.g., exact date and time when the slide was scanned). In a non-limiting example, a plurality of slides may be stored in a chronological order within a slide storage after being scanned. In some cases, 152 may include a string describing a scanner identification (ID); for instance, and without limitation, details about imaging device used, including model, manufacturer, and/or any unique identification number may be encoded and incorporated into metadata 152.

Still referring to FIG. 1, in some embodiments, metadata 152 associated with an image may include data such as image resolution (i.e., pixel density of slide image, measured in dots per inch [DPI]). Image resolution may indicate a level of detail captured in the associated slide image. In some cases, metadata 152 may include a bit depth (i.e., number of bits of information stored for each pixel), for example, and without limitation, a range of colors or shades of gray that can be represented may be determined by computing device 116 as a function of bit depth. Additionally, or additionally, in some cases, scanning metadata may include color profile (i.e., used color space) such as, without limitation, sRGB, RGB, RYB, CMY, CMYK, HSL, HSV or the like.

Still referring to FIG. 1, metadata 152 associated with an image may include data related to compression details e.g., details about compression algorithm, compression ratio, and/or the like. In some cases, computing device 116 may be configured to compress a generated slide image during scanning one or more slides. In an embodiment, computing device 116 may encode an image to reduce the file size and storage requirements while maintaining the essential visual information needed for further processing steps as described below. In an embodiment, compression and/or encoding of slide images may facilitate faster transmission of images. In some cases, computing device 116 may implement one or more lossless compression algorithms (i.e., maintain the original image quality of slide image), e.g., Huffman coding, Lempel-Ziv-Welch (LZW), Run-Length Encoding (RLE), and/or the like to identify and remove redundancy in each slide image without losing any information. In an embodiment, compressing and/or encoding each slide image May include converting the file format of each slide image into PNG, GIF, lossless JPEG2000 or the like. In other cases, one or more lossy compression algorithms such as, without limitation, Discrete Cosine Transform (DCT) in JPEG or Wavelet Transform in JPEG2000, may be implemented by computing device to compress and/or encoding each slide image with a higher compression ratio but a reduced image quality (i.e., discard some less significant information within each slide image, resulting in a smaller file size).

Still referring to FIG. 1, in some cases, metadata 152 associated an image may include a value representing a scan mode e.g., grayscale, color, or any other mode. In some cases, metadata 152 may also include image settings, for example, exposure settings containing details about light exposure during the scanning process, which can affect the brightness and contrast of an image. In some cases, metadata 152 may include imaging device settings. In an embodiment, metadata 152 may include one or more focus parameters. In a non-limiting example, information about focus setting may include focus distance, Z-stack information, focus offset, lens specification, correction data, and/or the like. In another embodiment, metadata 152 may include magnification level e.g., level of magnification used during scanning. In a further embodiment, if applicable, scan duration i.e., time taken to complete a scan of a slide may be determined and/or recorded. Metadata 152 including scan duration may be indicative or selected imaging device's depth or quality.

Still referring to FIG. 1, in some embodiments, metadata 152 associated with an image may include post-processing information. In a non-limiting example, any modifications or enhancements made to slide image after its being generated, such as brightness adjustments, contrast enhancements, or noise reduction. In some cases, scanning metadata may include slide label or slide identifier, for example, a unique identifier or label associated with a slide being scanned, may be incorporated into metadata 152 aiding in cataloging and retrieval as described herein. Additionally, or alternatively, operator details such as, without limitation, information related to human operator or system administrator responsible for any processing steps as described herein may be included in metadata 152, such as for accountability and/or quality control. In some cases, metadata 152 may include calibration data i.e., information about any calibration performed on imaging device prior to the slide scanning. In other cases, metadata 152 may further include environmental condition data (i.e., details about surrounding environment during scanning); for instance, and without limitation, temperature level, humidity level, and/or the like.

Still referring to FIG. 1, in some embodiments, metadata 152 associated with an image may include one or more error logs. In some cases, metadata 152 may include any errors or issues encountered during the scanning process, along with potential resolutions or notes.

Still referring to FIG. 1, in some embodiments, system 100 determines first scanning parameter 156 as a function of metadata 152. As used herein, a “scanning parameter” is a data structure which determines a setting at which a device captures an image, a data structure which determines whether or not an image of a z-stack is saved, or both. In non-limiting examples, a scanning parameter may include a magnification used to capture an image, an (x,y) coordinate set used to capture an image, an aperture used to capture an image, a shutter speed used to capture an image, a focus distance used to capture an image, which of a plurality of optical sensors is used to capture an image, and/or a datum indicating whether or not an image of a z-stack is to be saved. In some embodiments, metadata 152 is used to select an algorithm, and this algorithm is used to determine first scanning parameter 156.

Still referring to FIG. 1, in some embodiments, an algorithm may be selected as a function of a user input. For example, a user may select preferences for which algorithms are selected and system 100 may select algorithm according to those preferences. In another example, algorithm may be selected as a function of historical user inputs. In some embodiments, algorithm may be selected using rule based decision making. Algorithm may be selected using a decision tree. Algorithm may be selected using a machine learning model, such as a machine learning model trained on past user inputs. In some embodiments, metadata may include an instruction as to an algorithm to select and/or apply to one or more images. In some cases, an algorithm may be selected as a function of metadata 152. For example, algorithm may be selected based on an annotation on slide 120. Such an annotation may be captured in first image 148 and read using an OCR process. An OCR output may be interpreted using a language model, in order to identify algorithm. An annotation may indicate which algorithm to apply, and system 100 may select that algorithm. For example, an annotation may say to identify cells of a particular type, and system 100 may select algorithm that identifies cells of that type. An annotation may identify a feature of a sample on slide 120, and algorithm may be selected based on that feature. For example, an annotation may indicate that a sample contains a particular type of cell, and algorithm may be selected such that it identifies cells of that type, identifies cells of other types, counts cells of that type, counts cells of other types, or the like. In another example, an annotation may indicate cells of a particular type, and a first algorithm may identify cells of that type, and a second algorithm may contribute to producing a clearer image of a section of slide 120 containing cells of that type. For example, algorithm may identify a region containing those cells as a region of interest, algorithm may identify z-stack data which should be retained in order to capture a clear image of a region of interest, or algorithm may produce an output configuring system 100 to capture a more zoomed in image of a region of interest containing cells of the desired type. In some embodiments, annotations on slide 120 may be converted into metadata associated with one or more images, and both images and metadata may be factors for selecting algorithm. In some embodiments, algorithm may be selected and/or run automatically. This may occur while system 100 is still capturing images. Output of algorithm may be used to improve further image capture. In some embodiments, multiple algorithms are selected. In some embodiments, an algorithm, metadata, image, and/or other component of system 100 may be consistent with any feature disclosed in U.S. patent application Ser. No. 18/392,520, filed on Dec. 21, 2023, and titled “SYSTEM AND METHODS FOR SLIDE IMAGING,” the entirety of which is hereby incorporated by reference.

Still referring to FIG. 1, system 100 may automatically select algorithm to apply to first image 148. Algorithm may be applied to first image 148 and/or additional images of a plurality of images. Algorithms which may be applied to first image 148 include, in non-limiting examples, artifact removal algorithms, image processing algorithms, algorithms for selecting a region of interest, image segmentation algorithms, focus pattern recognition algorithms, feature detection algorithms, color correction algorithms, 3D reconstruction algorithms, quantitative analysis algorithms, and image classification algorithms. Non-limiting examples of feature detection algorithms include nuclei detection, cell detection, debris detection, cell type detection, and tissue type detection.

Still referring to FIG. 1, in some embodiments, a machine vision system and/or an optical character recognition system may be selected. In some embodiments, a machine vision system and/or an optical character recognition system may be used to determine one or more features of sample and/or slide 120. In a non-limiting example, an optical character recognition system may be used to identify writing on slide 120.

Still referring to FIG. 1, in some embodiments, system 100 may capture a plurality of images at differing sample focus depths. As used herein, a “sample focus depth” is a depth within a sample that an optical system is in focus. As used herein, a “focus distance” is an object side focal length. In some embodiments, first image and second image may have different focus distances and/or sample focus depths.

Still referring to FIG. 1, in some embodiments, system 100 may include a machine vision system. In some embodiments, a machine vision system may include at least a camera. A machine vision system may use images, such as images from at least a camera, to make a determination about a scene, space, and/or object. For example, in some cases a machine vision system may be used for world modeling or registration of objects within a space. In some cases, registration may include image processing, such as without limitation object recognition, feature detection, edge/corner detection, and the like. Non-limiting example of feature detection may include scale invariant feature transform (SIFT), Canny edge detection, Shi Tomasi corner detection, and the like. In some cases, registration may include one or more transformations to orient a camera frame (or an image or video stream) relative a three-dimensional coordinate system; exemplary transformations include without limitation homography transforms and affine transforms. In an embodiment, registration of first frame to a coordinate system may be verified and/or corrected using object identification and/or computer vision, as described above. For instance, and without limitation, an initial registration to two dimensions, represented for instance as registration to the x and y coordinates, may be performed using a two-dimensional projection of points in three dimensions onto a first frame, however. A third dimension of registration, representing depth and/or a z axis, may be detected by comparison of two frames; for instance, where first frame includes a pair of frames captured using a pair of cameras (e.g., stereoscopic camera also referred to in this disclosure as stereo-camera), image recognition and/or edge detection software may be used to detect a pair of stereoscopic views of images of an object; two stereoscopic views may be compared to derive z-axis values of points on object permitting, for instance, derivation of further z-axis points within and/or around the object using interpolation. This may be repeated with multiple objects in field of view, including without limitation environmental features of interest identified by object classifier and/or indicated by an operator. In an embodiment, x and y axes may be chosen to span a plane common to two cameras used for stereoscopic image capturing and/or an xy plane of a first frame; a result, x and y translational components and ϕ may be pre-populated in translational and rotational matrices, for affine transformation of coordinates of object, also as described above. Initial x and y coordinates and/or guesses at transformational matrices may alternatively or additionally be performed between first frame and second frame, as described above. For each point of a plurality of points on object and/or edge and/or edges of object as described above, x and y coordinates of a first stereoscopic frame may be populated, with an initial estimate of z coordinates based, for instance, on assumptions about object, such as an assumption that ground is substantially parallel to an xy plane as selected above. Z coordinates, and/or x, y, and z coordinates, registered using image capturing and/or object identification processes as described above may then be compared to coordinates predicted using initial guess at transformation matrices; an error function may be computed using by comparing the two sets of points, and new x, y, and/or z coordinates, may be iteratively estimated and compared until the error function drops below a threshold level. In some cases, a machine vision system may use a classifier, such as any classifier described throughout this disclosure. In some cases, a machine vision system may include a feature detection system, such as a deep learning feature detection system. In some embodiments, a machine vision system may include an edge detection system, a corner detection system, a blob detection system, and/or a ridge detection system. In non-limiting examples, a machine vision system may include a histogram of oriented gradients feature descriptor, a speeded up robust features feature descriptor, and/or a local binary pattern feature descriptor. In some embodiments, one or more layers of a deep neural network may implicitly act as a feature detection system.

Still referring to FIG. 1, in some embodiments, system 100 may identify a biological sample feature using a feature detection algorithm. In a non-limiting example, a feature detection algorithm may be trained to detect cells and/or tissues of a particular type. In another non-limiting example, a feature detection algorithm may be trained to detect nuclei of cells. In some embodiments, system 100 may select a feature detection algorithm as a function of metadata 152. In a non-limiting example, metadata 152 may indicate that a particular stain has been applied to a sample on slide 120, and a feature detection algorithm may be selected which recognizes cells, tissues, organelles, or the like highlighted by the stain. In another non-limiting example, metadata 152 may indicate a particular cell type and/or tissue type, and a feature detection algorithm may be selected which recognizes such cell type and/or tissue type. In another non-limiting example, metadata 152 may indicate a level of zoom and/or resolution of an image, and a feature detection algorithm optimized for such level of zoom and/or resolution may be selected. In some embodiments, system 100 may display a biological sample feature to a user. For example, system 100 may display a biological sample feature using user interface 140. In some embodiments, a feature detection algorithm may include a trained deep neural network.

Still referring to FIG. 1, in some embodiments, system 100 may store a biological sample feature and/or an image which a biological sample feature is determined based on (such as first image 148) in memory. Such memory may include, in non-limiting examples, memory 108 and/or memory of a database communicatively connected to system 100. In some embodiments, a biological sample feature and first image 148 may be stored in a manner such that a position of the biological sample feature within first image 148 is saved. In some embodiments, a biological sample feature and first image 148 may be displayed to a user. In a non-limiting example, a biological sample feature may be mapped to a particular point and/or region of first image 148, such that system 100 may display to a user first image 148 with the biological sample feature overlayed on first image 148.

Still referring to FIG. 1, in some embodiments, system 100 may remove an artifact from an image. As used herein, an “artifact” is a visual inaccuracy, an element of an image that distracts from an element of interest, an element of an image that obscures an element of interest, or another undesirable element of an image.

Still referring to FIG. 1, system 100 may include an image processing module. As used in this disclosure, an “image processing module” is a component designed to process digital images. In an embodiment, image processing module may include a plurality of software algorithms that can analyze, manipulate, or otherwise enhance an image, such as, without limitation, a plurality of image processing techniques as described below. In another embodiment, image processing module may include hardware components such as, without limitation, one or more graphics processing units (GPUs) that can accelerate the processing of large amount of images. In some cases, image processing module may be implemented with one or more image processing libraries such as, without limitation, OpenCV, PIL/Pillow, ImageMagick, and the like.

Still referring to FIG. 1, image processing module may be configured to receive images from optical sensor 124. One or more images may be transmitted, from optical sensor 124 to image processing module, via any suitable electronic communication protocol, including without limitation packet-based protocols such as transfer control protocol-internet protocol (TCP-IP), file transfer protocol (FTP) or the like. Receiving images may include retrieval of images from a data store containing images as described below; for instance, and without limitation, images may be retrieved using a query that specifies a timestamp that images may be required to match.

Still referring to FIG. 1, image processing module may be configured to process images. In an embodiment, image processing module may be configured to compress and/or encode images to reduce the file size and storage requirements while maintaining the essential visual information needed for further processing steps as described below. In an embodiment, compression and/or encoding of an image may facilitate faster transmission of images. In some cases, image processing module may be configured to perform a lossless compression on images, wherein the lossless compression may maintain the original image quality of images. In a non-limiting example, image processing module may utilize one or more lossless compression algorithms, such as, without limitation, Huffman coding, Lempel-Ziv-Welch (LZW), Run-Length Encoding (RLE), and/or the like to identify and remove redundancy in images without losing any information. In such embodiment, compressing and/or encoding each image of images may include converting the file format of each image into PNG, GIF, lossless JPEG2000 or the like. In an embodiment, images compressed via lossless compression may be perfectly reconstructed to the original form (e.g., original image resolution, dimension, color representation, format, and the like) of images. In other cases, image processing module may be configured to perform a lossy compression on images, wherein the lossy compression may sacrifice some image quality of images to achieve higher compression ratios. In a non-limiting example, image processing module may utilize one or more lossy compression algorithms, such as, without limitation, Discrete Cosine Transform (DCT) in JPEG or Wavelet Transform in JPEG2000, discard some less significant information within images, resulting in a smaller file size but a slight loss of image quality of images. In such embodiment, compressing and/or encoding images may include converting the file format of each image into JPEG, WebP, lossy JPEG2000, or the like.

Still referring to FIG. 1, in an embodiment, processing images may include determining a degree of quality of depiction of a region of interest of an image. In an embodiment, image processing module may determine a degree of blurriness of images. In a non-limiting example, image processing module may perform a blur detection by taking a Fourier transform, or an approximation such as a Fast Fourier Transform (FFT) of images and analyzing a distribution of low and high frequencies in the resulting frequency-domain depiction of images; for instance, and without limitation, numbers of high-frequency values below a threshold level may indicate blurriness. In another non-limiting example, detection of blurriness may be performed by convolving images, a channel of images, or the like with a Laplacian kernel; for instance, and without limitation, this may generate a numerical score reflecting a number of rapid changes in intensity shown in each image, such that a high score indicates clarity and a low score indicates blurriness. In some cases, blurriness detection may be performed using a Gradient-based operator, which measures operators based on the gradient or first derivative of images, based on the hypothesis that rapid changes indicate sharp edges in the image, and thus are indicative of a lower degree of blurriness. In some cases, blur detection may be performed using Wavelet-based operator, which takes advantage of the capability of coefficients of the discrete wavelet transform to describe the frequency and spatial content of images. In some cases, blur detection may be performed using statistics-based operators take advantage of several image statistics as texture descriptors in order to compute a focus level. In other cases, blur detection may be performed by using discrete cosine transform (DCT) coefficients in order to compute a focus level of images from its frequency content. Additionally, or alternatively, image processing module may be configured to rank images according to degree of quality of depiction of a region of interest and select a highest-ranking image from a plurality of images.

Still referring to FIG. 1, processing images may include enhancing an image or at least a region of interest via a plurality of image processing techniques to improve the quality (or degree of quality of depiction) of an image for better processing and analysis as described further in this disclosure. In an embodiment, image processing module may be configured to perform a noise reduction operation on an image, wherein the noise reduction operation may remove or minimize noise (arises from various sources, such as sensor limitations, poor lighting conditions, image compression, and/or the like), resulting in a cleaner and more visually coherent image. In some cases, noise reduction operation may be performed using one or more image filters; for instance, and without limitation, noise reduction operation may include Gaussian filtering, median filtering, bilateral filtering, and/or the like. Noise reduction operation may be done by image processing module, by averaging or filtering out pixel values in neighborhood of each pixel of an image to reduce random variations.

Still referring to FIG. 1, in another embodiment, image processing module may be configured to perform a contrast enhancement operation on an image. In some cases, an image may exhibit low contrast, which may, for example, make a feature difficult to distinguish from the background. Contrast enhancement operation may improve the contrast of an image by stretching the intensity range of the image and/or redistributing the intensity values (i.e., degree of brightness or darkness of a pixel in the image). In a non-limiting example, intensity value may represent the gray level or color of each pixel, scale from 0 to 255 in intensity range for an 8-bit image, and scale from 0 to 16,777,215 in a 24-bit color image. In some cases, contrast enhancement operation may include, without limitation, histogram equalization, adaptive histogram equalization (CLAHE), contrast stretching, and/or the like. Image processing module may be configured to adjust the brightness and darkness levels within an image to make a feature more distinguishable (i.e., increase degree of quality of depiction). Additionally, or alternatively, image processing module may be configured to perform a brightness normalization operation to correct variations in lighting conditions (i.e., uneven brightness levels). In some cases, an image may include a consistent brightness level across a region after brightness normalization operation performed by image processing module. In a non-limiting example, image processing module may perform a global or local mean normalization, where the average intensity value of an entire image or region of an image may be calculated and used to adjust the brightness levels.

Still referring to FIG. 1, in other embodiments, image processing module may be configured to perform a color space conversion operation to increase degree of quality of depiction. In a non-limiting example, in case of a color image (i.e., RGB image), image processing module may be configured to convert RGB image to grayscale or HSV color space. Such conversion may emphasize the differences in intensity values between a region or feature of interest and the background. Image processing module may further be configured to perform an image sharpening operation such as, without limitation, unsharp masking, Laplacian sharpening, high-pass filtering, and/or the like. Image processing module may use image sharpening operation to enhance the edges and fine details related to a region or feature of interest within an image by emphasizing high-frequency components within an image.

Still referring to FIG. 1, processing images may include isolating a region or feature of interest from the rest of an image as a function of plurality of image processing techniques. Images may include highest-ranking image selected by image processing module as described above. In an embodiment, plurality of image processing techniques may include one or more morphological operations, wherein the morphological operations are techniques developed based on set theory, lattice theory, topology, and random functions used for processing geometrical structures using a structuring element. A “structuring element,” for the purpose of this disclosure, is a small matrix or kernel that defines a shape and size of a morphological operation. In some cases, structing element may be centered at each pixel of an image and used to determine an output pixel value for that location. In a non-limiting example, isolating a region or feature of interest from an image may include applying a dilation operation, wherein the dilation operation is a basic morphological operation configured to expand or grow the boundaries of objects (e.g., a cell, a dust particle, and the like) in an image. In another non-limiting example, isolating a region or feature of interest from an image may include applying an erosion operation, wherein the erosion operation is a basic morphological operation configured to shrink or erode the boundaries of objects in an image. In another non-limiting example, isolating a region or feature of interest from an image may include applying an opening operation, wherein the opening operation is a basic morphological operation configured to remove small objects or thin structures from an image while preserving larger structures. In a further non-limiting example, isolating a region or feature of interest from an image may include applying a closing operation, wherein the closing operation is a basic morphological operation configured to fill in small gaps or holes in objects in an image while preserving the overall shape and size of the objects. These morphological operations may be performed by image processing module to enhance the edges of objects, remove noise, or fill gaps in a region or feature of interest before further processing.

Still referring to FIG. 1, in an embodiment, isolating a region or feature of interest from an image may include utilizing an edge detection technique, which may detect one or more shapes defined by edges. An “edge detection technique,” as used in this disclosure, includes a mathematical method that identifies points in a digital image, at which the image brightness changes sharply and/or has a discontinuity. In an embodiment, such points may be organized into straight and/or curved line segments, which may be referred to as “edges.” Edge detection technique may be performed by image processing module, using any suitable edge detection algorithm, including without limitation Canny edge detection, Sobel operator edge detection, Prewitt operator edge detection, Laplacian operator edge detection, and/or Differential edge detection. Edge detection technique may include phase congruency-based edge detection, which finds all locations of an image where all sinusoids in the frequency domain, for instance as generated using a Fourier decomposition, may have matching phases which may indicate a location of an edge. Edge detection technique may be used to detect a shape of a feature of interest such as a cell, indicating a cell membrane or wall; in an embodiment, edge detection technique may be used to find closed figures formed by edges.

Still referring to FIG. 1, in a non-limiting example, isolating a feature of interest from an image may include determining a feature of interest via edge detection technique. A feature of interest may include a specific area within a digital image that contains information relevant to further processing as described below. In a non-limiting example, an image data located outside a feature of interest may include irrelevant or extraneous information. Such portion of an image containing irrelevant or extraneous information may be disregarded by image processing module, thereby allowing resources to be concentrated at a feature of interest. In some cases, feature of interest may vary in size, shape, and/or location within an image. In a non-limiting example feature of interest may be presented as a circle around the nucleus of a cell. In some cases, feature of interest may specify one or more coordinates, distances and the like, such as center and radius of a circle around the nucleus of a cell in an image. Image processing module may then be configured to isolate feature of interest from the image based on feature of interest. In a non-limiting example, image processing module may crop an image according to a bounding box around a feature of interest.

Still referring to FIG. 1, image processing module may be configured to perform a connected component analysis (CCA) on an image for feature of interest isolation. As used in this disclosure, a “connected component analysis (CCA),” also known as connected component labeling, is an image processing technique used to identify and label connected regions within a binary image (i.e., an image which each pixel having only two possible values: 0 or 1, black or white, or foreground and background). “Connected regions,” as described herein, is a group of adjacent pixels that share the same value and are connected based on a predefined neighborhood system such as, without limitation, 4-connected or 8-connected neighborhoods. In some cases, image processing module may convert an image into a binary image via a thresholding process, wherein the thresholding process may involve setting a threshold value that separates the pixels of an image corresponding to feature of interest (foreground) from those corresponding to the background. Pixels with intensity values above the threshold may be set to 1 (white) and those below the threshold may be set to 0 (black). In an embodiment, CCA may be employed to detect and extract feature of interest by identifying a plurality of connected regions that exhibit specific properties or characteristics of the feature of interest. Image processing module may then filter plurality of connected regions by analyzing plurality of connected regions properties such as, without limitation, area, aspect ratio, height, width, perimeter, and/or the like. In a non-limiting example, connected components that closely resemble the dimensions and aspect ratio of feature of interest may be retained, by image processing module as feature of interest, while other components may be discarded. Image processing module may be further configured to extract feature of interest from an image for further processing as described below.

Still referring to FIG. 1, in an embodiment, isolating feature of interest from an image may include segmenting a region depicting a feature of interest into a plurality sub-regions. Segmenting a region into sub-regions may include segmenting a region as a function of feature of interest and/or CCA via an image segmentation process. As used in this disclosure, an “image segmentation process” is a process for partition a digital image into one or more segments, where each segment represents a distinct part of the image. Image segmentation process may change the representation of images. Image segmentation process may be performed by image processing module. In a non-limiting example, image processing module may perform a region-based segmentation, wherein the region-based segmentation involves growing regions from one or more seed points or pixels on an image based on a similarity criterion. Similarity criterion may include, without limitation, color, intensity, texture, and/or the like. In a non-limiting example, region-based segmentation may include region growing, region merging, watershed algorithms, and the like.

Still referring to FIG. 1, in some embodiments, system 100 may remove an artifact identified by a machine vision system or an optical character recognition system, which are described above. Non-limiting examples of artifacts that may be removed include dust particles, bubbles, cracks in slide 120, writing on slide 120, shadows, visual noise such as in a grainy image, and the like. In some embodiments, an artifact may be partially removed and/or lowered in visibility.

Still referring to FIG. 1, in some embodiments, an artifact may be removed using an artifact removal machine learning model. In some embodiments, artifact removal machine learning model may be trained on a dataset including images, associated with images without artifacts. In some embodiments, artifact removal machine learning model may accept as an input an image including an artifact and may output an image without the artifact. For example, artifact removal machine learning model may accept as an input an image including a bubble in a slide and may output an image that does not include the bubble. In some embodiments, artifact removal machine learning model may include a generative machine learning model such as a diffusion model. A diffusion model may learn the structure of a dataset by modeling the way data points diffuse through a latent space. In some embodiments, artifact removal may be done locally. For example, system 100 may include an already trained artifact removal machine learning model and may apply the model to an image. In some embodiments, artifact removal may be done externally. For example, system 100 may transmit image data to another computing device and may receive an image with an artifact removed. In some embodiments, an artifact may be removed in real time. In some embodiments, an artifact may be removed based on identification by a user. For example, a user may drag a box around an artifact using a mouse cursor, and system 100 may remove an artifact in the box.

Still referring to FIG. 1, in some embodiments, an algorithm which selects a region of interest, detects a focus pattern, and/or determines which regions of interest contain a sample may be selected. Such algorithms may be consistent with algorithms described in U.S. patent application Ser. No. 18/384,840, filed on Oct. 28, 2023, and titled “APPARATUS AND METHODS FOR SLIDE IMAGING,” the entirety of which is incorporated herein by reference.

Still referring to FIG. 1, in some embodiments, first scanning parameter 156 may include a coordinate set, and capturing a z-stack as a function of first scanning parameter 156 includes capturing the z-stack at coordinates of the coordinate set. Such a coordinate set may include an (x,y) coordinate set identifying a location on slide 120. Such a coordinate set may further include a focus distance and/or sample focus depth. Such a coordinate set may be determined, for example, from metadata indicating a tissue type of a biological sample on slide 120. In some embodiments, biological samples of different tissue types may have different thicknesses, such that a z-stack at a top of a sample is captured at different focus distances depending on the tissue type. As another example, a coordinate set may be determined from metadata indicating a medical condition of a subject from which a biological sample was taken and/or a stain type applied to slide 120. In some embodiments, such metadata may be obtained from an electronic health record as described above. Such metadata may be used to determine which locations on slide 120 are of particular interest using a machine vision system, and a z-stack may be captured at such a location.

Still referring to FIG. 1, in some embodiments, first scanning parameter 156 may include a magnification, and capturing a z-stack as a function of first scanning parameter 156 includes capturing the z-stack at the magnification. Such a magnification may be determined, for example, from metadata indicating a medical condition of a subject from which a biological sample was taken. For example, different magnification images may be useful for analyzing features of samples of subjects with different medical conditions (for example because scientists may be looking for features of different sizes), and a scanning process may use the same magnification to capture a z-stack as the magnification used to capture an image based on the z-stack.

Still referring to FIG. 1, in some embodiments, first scanning parameter 156 may include a z-stack size and capturing a z-stack as a function of first scanning parameter 156 includes capturing the z-stack such that the z-stack includes a number of images according to the z-stack size. Such a z-stack size may be determined, for example, from metadata indicating a tissue type of a biological sample on slide 120. In some embodiments, biological samples of different tissue types may have different variability in thickness. The degree of variability in thickness may correspond to a number of images to be captured in a z-stack. For example, a z-stack may include more images in a high thickness variability sample than in a low thickness variability sample, in order to cover a larger vertical distance. Similarly, first scanning parameter 156 may include a z-step size and capturing a z-stack as a function of first scanning parameter 156 includes capturing the z-stack such that the z-stack has a distance between focus distances of images of the z-stack according to and/or defined by the z-step size. A higher z-step size may be used to capture a z-stack of a sample with a higher thickness variability than a sample with a low thickness variability. In another example, z-stack size and/or z-step size may be modified as a function of the variability in height within a sample a feature of interest is positioned.

Still referring to FIG. 1, in some embodiments, first scanning parameter 156 may include a fusion algorithm parameter and capturing a z-stack as a function of first scanning parameter 156 includes capturing the z-stack at coordinates determined using a fusion algorithm. As used herein, a “fusion algorithm” is an algorithm that combines data from a plurality of images with overlapping fields of view. For example, a fusion algorithm parameter may include a relative position of optical sensors used to capture the plurality of images, and this may be determined as a function of metadata indicating which optical sensors of an optical system were used to capture the plurality of images.

Still referring to FIG. 1, in some embodiments, first scanning parameter 156 may include a z-stack preservation datum and computing device 116 may determine whether to store one or more images of a z-stack in a repository as a function of the z-stack preservation datum. As used herein, a “z-stack preservation datum” is a data structure indicating whether an image of a z-stack is stored, which type of memory is used to store an image of a z-stack, or both. For example, metadata indicating a medical condition of a subject from which a biological sample was taken may be used to identify a region of interest (such as based on a machine vision analysis of which regions of slide 120 contain sample relevant to a medical condition such as a relevant cell type), and whether or not a z-stack is within this region of interest may be used to determine a z-stack preservation datum. A repository may include computer memory such as memory 108 and/or an external database.

Still referring to FIG. 1, in some embodiments, system 100 may capture z-stack 160 as a function of first scanning parameter 156. As used herein, a “z-stack” is a set of images captured at varying focus distances. In some embodiments, images of a z-stack may have a consistent step size between adjacent images of the z-stack. In some embodiments, images of a z-stack may be captured at the same (x,y) coordinates of a slide. Such (x,y) coordinates may indicate horizontal coordinates and/or coordinates on a plane perpendicular to the direction an optical sensor used to capture a z-stack is pointing while capturing the z-stack. Non-limiting examples of settings at which z-stack 160 may be captured are described above in the context of first scanning parameter 156.

Still referring to FIG. 1, in some embodiments, system 100 may identify a second scanning parameter 164 as a function of z-stack 160. In some embodiments, second scanning parameter 164 may include a focus distance and/or sample focus depth used to capture second image 168. In some embodiments, one or more other forms of second scanning parameter 164 may be determined as a function of z-stack 160, as described in the context of first scanning parameter 156. In some embodiments, system 100 may capture second image 168 as a function of second scanning parameter 164. For example, a focus distance and/or sample focus depth according to second scanning parameter 164 may be used.

Still referring to FIG. 1, in some embodiments, system 100 may, using an optical system, capture a plurality of images of slide 120 and assemble second image 168 from the plurality of images. For example, a stitching algorithm may be used to assemble second image 168 from such a plurality of images. In some embodiments, one or more images of a plurality of images used to assemble second image 168 may be captured according to second scanning parameter 164. For example, one or more images of a plurality of images used to assemble second image 168 may be captured using a focus distance and/or sample focus depth according to second scanning parameter 164.

Still referring to FIG. 1, in some embodiments, system 100 may determine a volumetric image set of slide 120. In some embodiments, a volumetric image set of slide 120 may be determined as a function of z-stack 160. As used herein, a “volumetric image set” is a plurality of images with overlapping fields of view where at least two images of the plurality of images have different focus distances. In some embodiments, images of a volumetric image set may have a consistent step size between focus distances of adjacent images. In some embodiments, one or more images of a volumetric image set may include image data of z-stack 160. For example, many z-stacks may be captured at different (x,y) points on slide 120, such as in a grid pattern. Such z-stacks may be captured using consistent scanning parameters such as magnification and z-step size. Images from such z-stacks may be combined together to create an image of a volumetric image set. For example, images from such z-stacks may be combined using a stitching algorithm as described herein. In some embodiments, an image of a volumetric image set includes image data from a plurality of z-stack images captured at different (x,y) points, and with the same focus distance. This may be used to create a volumetric image set including a plurality of images with different focus distances, each covering a particular region of slide 120 (such as a region of interest), an entirety of a sample on slide 120, and/or an entirety of slide 120. In some embodiments, one or more images of volumetric image set may be determined using a fusion algorithm.

Still referring to FIG. 1, in some embodiments, a volumetric image set of slide 120 may be determined as a function of second scanning parameter 164. For example, volumetric image set may be assembled from a plurality of images, where one or more such images are captured as a function of second scanning parameter 164.

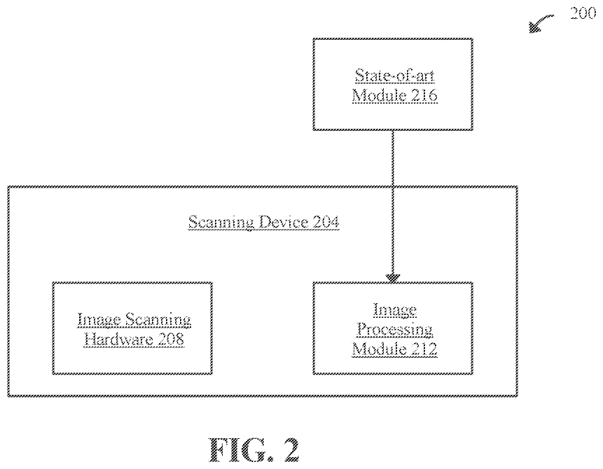

Referring now to FIG. 2, a block diagram of an exemplary embodiment of system 200 is provided. System 200 may include scanning device 204. Image scanning hardware 208 may include an image processing module 212 that can be updated with latest state-of-art modules 216. Additionally, algorithms and/or machine learning models fit to identify features in the slide to be scanned may be downloaded. For example, an algorithm and/or machine learning model described above may be downloaded. In some embodiments, scanning device 204 may be configured to download an algorithm and/or machine learning model when metadata is available, such as due to a presence of an annotation on a slide or through other media.

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm instantiated in hardware or software logic, data structures, and/or functions that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure. As a non-limiting illustrative example, inputs may include image data and outputs may include glyphs as in optical character recognition.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 300 may generate a classifier using a classification algorithm, defined as a processes whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data to particular glyphs.

With further reference to FIG. 3, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Still referring to FIG. 3, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value.

As a non-limiting example, and with further reference to FIG. 3, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 3, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 3, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning models 324. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include image data as described above as inputs, glyphs as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

With further reference to FIG. 3, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 3, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 332 may not require a response variable; unsupervised processes 332 may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task clastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 3, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 3, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 3, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 3, one or more processes or algorithms described above may be performed by at least a dedicated hardware unit 336. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 336 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 336 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 336 to perform one or more operations described herein, such as evaluation of model and/or algorithm outputs, one-time or iterative updates to parameters, coefficients, weights, and/or biases, and/or any other operations such as vector and/or matrix operations as described in this disclosure.

With continued reference to FIG. 3, system 100 may use user feedback to train the machine-learning models and/or classifiers described above. For example, classifier may be trained using past inputs and outputs of classifier. In some embodiments, if user feedback indicates that an output of classifier was “bad,” then that output and the corresponding input may be removed from training data used to train classifier, and/or may be replaced with a value entered by, e.g., another user that represents an ideal output given the input the classifier originally received, permitting use in retraining, and adding to training data; in either case, classifier may be retrained with modified training data as described in further detail below. In some embodiments, training data of classifier may include user feedback.

With continued reference to FIG. 3, in some embodiments, an accuracy score may be calculated for classifier using user feedback. For the purposes of this disclosure, “accuracy score,” is a numerical value concerning the accuracy of a machine-learning model. For example, a plurality of user feedback scores may be averaged to determine an accuracy score. In some embodiments, a cohort accuracy score may be determined for particular cohorts of persons. For example, user feedback for users belonging to a particular cohort of persons may be averaged together to determine the cohort accuracy score for that particular cohort of persons and used as described above. Accuracy score or another score as described above may indicate a degree of retraining needed for a machine-learning model such as a classifier; system 100 may perform a larger number of retraining cycles for a higher number (or lower number, depending on a numerical interpretation used), and/or may collect more training data for such retraining, perform more training cycles, apply a more stringent convergence test such as a test requiring a lower mean squared error, and/or indicate to a user and/or operator that additional training data is needed.

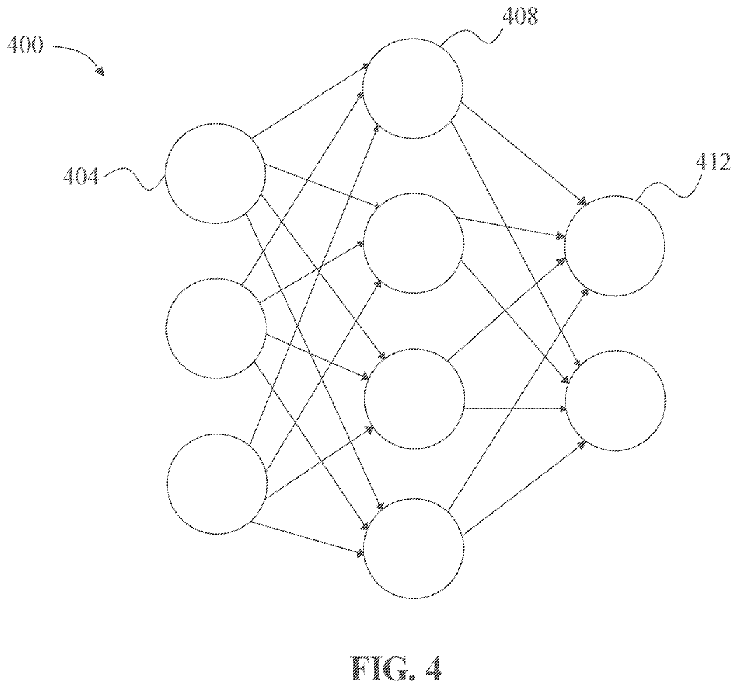

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

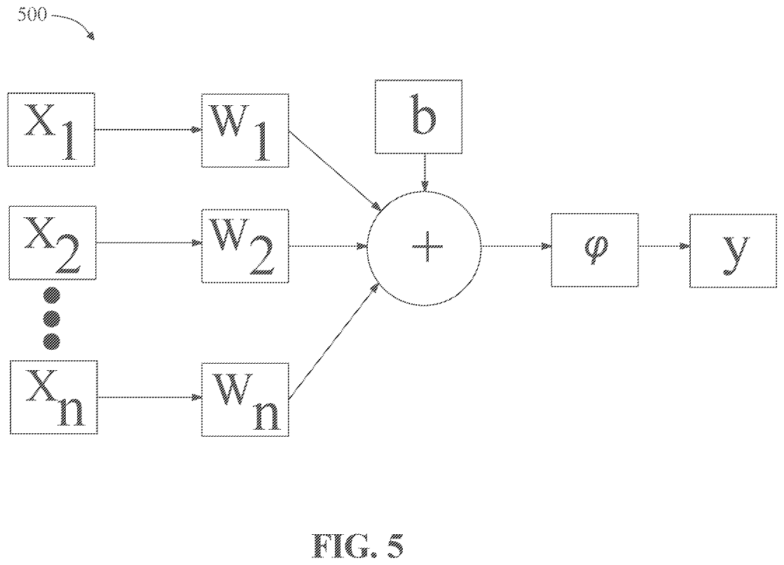

Referring now to FIG. 5, an exemplary embodiment of a node 500 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max (0, x), a “leaky” and/or “parametric” rectified linear unit function such as f(x)=max(ax, x) for some a, an exponential linear units function such as

$$f ( x ) = \{ \begin{matrix}x & for x \geq 0 \\ α ( {e}^{x} - 1 ) & for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) & for x < 0 \\ x & for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

Still referring to FIG. 5, a “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like. CNN may include, without limitation, a deep neural network (DNN) extension, where a DNN is defined as a neural network with two or more hidden layers.

Still referring to FIG. 5, in some embodiments, a convolutional neural network may learn from images. In non-limiting examples, a convolutional neural network may perform tasks such as classifying images, detecting objects depicted in an image, segmenting an image, and/or processing an image. In some embodiments, a convolutional neural network may operate such that each node in an input layer is only connected to a region of nodes in a hidden layer. In some embodiments, the regions in aggregate may create a feature map from an input layer to the hidden layer. In some embodiments, a convolutional neural network may include a layer in which the weights and biases for all nodes are the same. In some embodiments, this may allow a convolutional neural network to detect a feature, such as an edge, across different locations in an image.

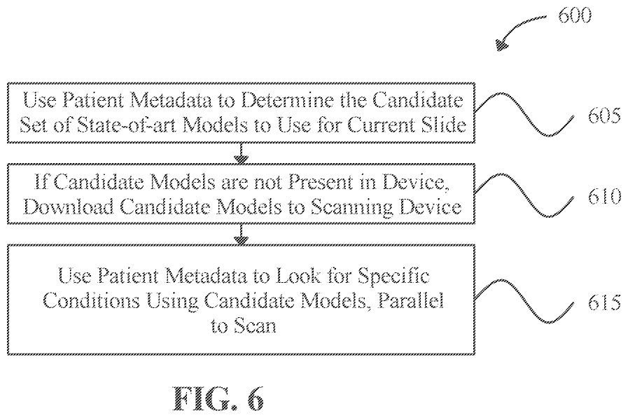

Referring now to FIG. 6, an exemplary embodiment of a method 600 of digitizing a slide is illustrated. Given patient information associated with the slide to be scanned, that information is used to create a candidate set of machine learning models that are best suited for both informing the scanning process as well as detect features specific to the patient condition that was derived from patient information 605. If the candidate models are not present on the device, they are downloaded to the device prior to scanning the slide 610.

Still referring to FIG. 6, a scanning process may be informed by features detected by such downloaded algorithms. This may include but is not limited to the resolution to be scanned, the regions to be scanned, and/or the number of stacks to be scanned 615.

Still referring to FIG. 6, in an embodiment of the invention, even when the hardware has sufficient memory to hold a repository of models, a software update process may facilitate updating a repository to have state-of-art models resident always on a device.

Still referring to FIG. 6, the presence of models looking for a specific condition given patient information enables multiple iterations of a scan on the device with different hardware and software settings until features indicating the presence of or absence of the specific condition above a confidence threshold. This eliminates slow cycles of multiple rescans initiated by two-stage processes. For instance, machine learning models detect features of regions that are being scanned. They aggregate information across regions to detect features which in turn are used to manage the scanning process-such as rescanning at a different magnification, resolution and illumination to detect certain features as and when required.

Still referring to FIG. 6, in another embodiment, where hardware has limited capabilities but has high bandwidth connectivity, machine learning models may be run outside the device. For example, machine learning models may be run local to the device but external to it. These models work in parallel to a scanning process and perform the same operations they would if they were locally resident on a scanning device.

Still referring to FIG. 6, in another embodiment of invention, stacks are captured and saved using the input of an inline software algorithm to get the entire feature of interest into focus. These saved stacks aids the user to view the features as a continuum diffusion process rather than as a single fused output where the information of refractile objects like fungal hyphae is lost, whereas when stacks/layers are viewed sequentially, the refractile objects are better appreciated and also give better control similar to fine focusing of microscope.

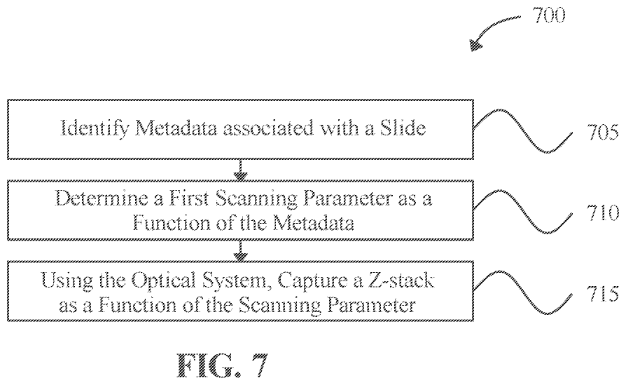

Referring now to FIG. 7, an exemplary embodiment of a method 700 of digitizing a slide is illustrated. One or more steps if method 700 may be implemented, without limitation, as described with reference to other figures. One or more steps of method 700 may be implemented, without limitation, using at least a processor.

Still referring to FIG. 7, in some embodiments, method 700 may include using an optical system, capturing a first image of a slide.

Still referring to FIG. 7, in some embodiments, method 700 may include identifying metadata associated with a slide. In some embodiments, identifying metadata of the first image comprises performing optical character recognition on the first image. In some embodiments, identifying the metadata includes capturing a first image of the slide; and identifying the metadata as a function of the first image by performing optical character recognition on the first image. In some embodiments, the slide includes a biological sample of a subject; and identifying the metadata comprises identifying a biological sample feature of the subject. In some embodiments, the slide comprises a biological sample of a subject; and identifying the metadata comprises receiving an electronic health record of the subject.

Still referring to FIG. 7, in some embodiments, method 700 may include determining a first scanning parameter as a function of the metadata 710. In some embodiments, the slide comprises a biological sample of a subject; and identifying metadata of the first image comprises identifying a biological sample feature from an electronic health record of the subject. In some embodiments, the first scanning parameter comprises a coordinate set; and capturing the z-stack as a function of the first scanning parameter comprises capturing the z-stack at coordinates of the coordinate set. In some embodiments, the first scanning parameter comprises a magnification; and capturing the z-stack as a function of the first scanning parameter comprises capturing the z-stack at the magnification. In some embodiments, the first scanning parameter comprises a z-stack size; and capturing the z-stack as a function of the first scanning parameter comprises capturing the z-stack such that the z-stack comprises a number of images according to the z-stack size. In some embodiments, the first scanning parameter comprises a z-step size; and capturing the z-stack as a function of the first scanning parameter comprises capturing the z-stack with a distance between focus distances of images of the z-stack according to the z-step size. In some embodiments, the first scanning parameter comprises a fusion algorithm parameter; and capturing the z-stack as a function of the first scanning parameter comprises capturing the z-stack at coordinates determined using a fusion algorithm. In some embodiments, the first scanning parameter comprises a z-stack preservation datum; and the method further comprises storing the z-stack as a function of the z-stack preservation datum.

Still referring to FIG. 7, in some embodiments, method 700 may include using the optical system, capturing a z-stack as a function of the first scanning parameter 715.

Still referring to FIG. 7, in some embodiments, method 700 may further include identifying a second scanning parameter as a function of the z-stack; using the optical system, capturing a plurality of images of the slide; and/or assembling a second image from the plurality of images. In some embodiments, method 700 may further include determining a volumetric image set of the slide as a function of the z-stack.

Still referring to FIG. 7, in some embodiments, method 700 may further include identifying a biological sample feature using a feature detection algorithm selected as a function of the metadata; and displaying the biological sample feature to a user. In some embodiments, identifying the metadata includes capturing a first image of the slide; the method further comprises identifying a biological sample feature using a feature detection algorithm selected as a function of the metadata; the method further comprises storing the biological sample feature and the first image in memory; and the method further comprises displaying the biological sample feature and the first image to a user. In some embodiments, the feature detection algorithm comprises a trained deep neural network.

A system or method described herein may be consistent with any system or method disclosed in U.S. patent application Ser. No. 18/598,307, filed on Mar. 7, 2024, and titled “APPARATUS AND METHODS FOR REAL-TIME IMAGE GENERATION,” the entirety of which is hereby incorporated by reference.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

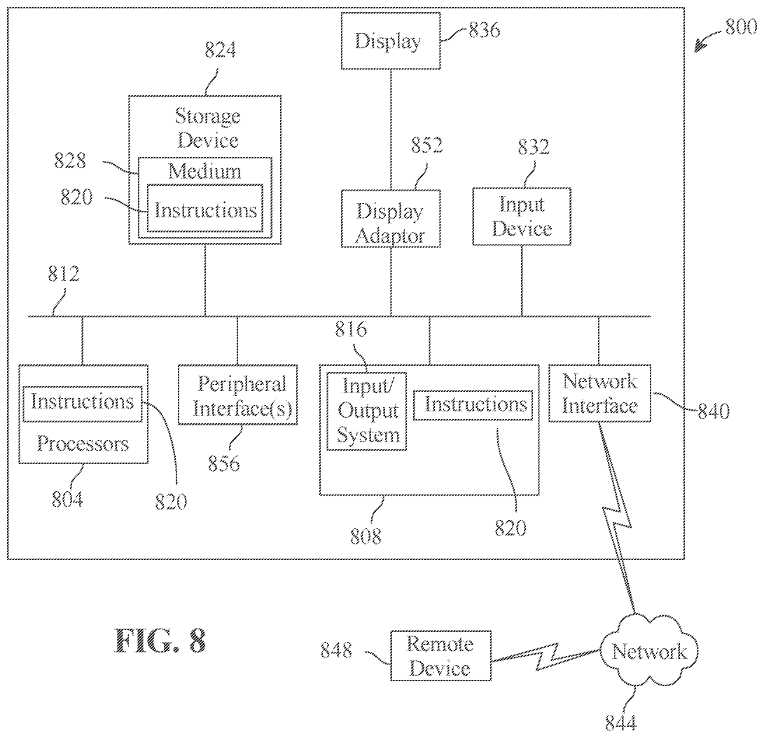

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for digitizing a slide, the system comprising:
a slide;
an optical system; and
a computing device configured to:
identify metadata associated with the slide;
determine a first scanning parameter as a function of the metadata; and
using the optical system, capture a z-stack as a function of the first scanning parameter.

2. The system of claim 1, wherein identifying the metadata comprises:
capturing a first image of the slide; and
identifying the metadata as a function of the first image by performing optical character recognition on the first image.

3. The system of claim 1, wherein:
the slide comprises a biological sample of a subject; and
identifying the metadata comprises receiving an electronic health record of the subject.

4. The system of claim 1, wherein the computing device is further configured to determine a volumetric image set of the slide as a function of the z-stack.

5. The system of claim 1, wherein:
the first scanning parameter comprises a coordinate set; and
capturing the z-stack as a function of the first scanning parameter comprises capturing the z-stack at coordinates of the coordinate set.

6. The system of claim 1, wherein:
the first scanning parameter comprises a magnification; and
capturing the z-stack as a function of the first scanning parameter comprises capturing the z-stack at the magnification.

7. The system of claim 1, wherein:
the first scanning parameter comprises a z-stack size; and
capturing the z-stack as a function of the first scanning parameter comprises capturing the z-stack such that the z-stack comprises a number of images according to the z-stack size.

8. The system of claim 1, wherein:
the first scanning parameter comprises a z-step size; and
capturing the z-stack as a function of the first scanning parameter comprises capturing the z-stack with a distance between focus distances of images of the z-stack defined by the z-step size.

9. The system of claim 1, wherein:
identifying the metadata comprises capturing a first image of the slide;
the computing device is configured to identify a biological sample feature using a feature detection algorithm selected as a function of the metadata;
the computing device is configured to store the biological sample feature and the first image in memory; and
the computing device is configured to display the biological sample feature and the first image to a user.

10. The system of claim 9, wherein the feature detection algorithm comprises a trained deep neural network.

11. A method of digitizing a slide, the method comprising:
using at least a processor, identifying metadata associated with the slide;
using the at least a processor, determining a first scanning parameter as a function of the metadata; and
using an optical system and the at least a processor, capturing a z-stack as a function of the first scanning parameter.

12. The method of claim 11, wherein identifying the metadata comprises:
capturing a first image of the slide; and
identifying the metadata as a function of the first image by performing optical character recognition on the first image.

13. The method of claim 11, wherein:
the slide comprises a biological sample of a subject; and
identifying the metadata comprises receiving an electronic health record of the subject.

14. The method of claim 11, wherein the method further comprises determining a volumetric image set of the slide as a function of the z-stack.

15. The method of claim 11, wherein:
the first scanning parameter comprises a coordinate set; and
capturing the z-stack as a function of the first scanning parameter comprises capturing the z-stack at coordinates of the coordinate set.

16. The method of claim 11, wherein:
the first scanning parameter comprises a magnification; and
capturing the z-stack as a function of the first scanning parameter comprises capturing the z-stack at the magnification.

17. The method of claim 11, wherein:
the first scanning parameter comprises a z-stack size; and
capturing the z-stack as a function of the first scanning parameter comprises capturing the z-stack such that the z-stack comprises a number of images according to the z-stack size.

18. The method of claim 11, wherein:
the first scanning parameter comprises a z-step size; and
capturing the z-stack as a function of the first scanning parameter comprises capturing the z-stack with a distance between focus distances of images of the z-stack defined by the z-step size.

19. The method of claim 11, wherein:
identifying the metadata comprises capturing a first image of the slide;
the method further comprises, using the at least a processor, identifying a biological sample feature using a feature detection algorithm selected as a function of the metadata;
the method further comprises, using the at least a processor, storing the biological sample feature and the first image in memory; and
the method further comprises, using the at least a processor, displaying the biological sample feature and the first image to a user.

20. The method of claim 19, wherein the feature detection algorithm comprises a trained deep neural network.
